(19) United States
(12) Patent Application Publication
(10) Pub. No.: US 2014/0142053 A1
(43) Pub. Date: May 22, 2014

(54) Use of a Composition Comprising Acylated Steryl Glucoside in the Manufacture of a Product

(71) Applicants: Maznah Ismail, Serdang (MY); Mustapha Umar Imam, Serdang (MY); Siti Nor Asma Musa, Serdang (MY)

(72) Inventors: Maznah Ismail, Serdang (MY); Mustapha Umar Imam, Serdang (MY); Siti Nor Asma Musa, Serdang (MY)

(73) Assignee: UNIVERSITI PUTRA MALAYSIA, Serdang (MY)

(21) Appl. No.: 14/118,931

(22) PCT Filed: Apr. 26, 2012
(86) PCT No.: PCT/MY2012/000091

§ 371 Date: Nov. 20, 2013

(30) Foreign Application Priority Data
May 20, 2011 (MY) PI 2011002256

(51) Int. Cl. A61K 31/7004; A61K 36/899

(52) U.S. Cl. CPC A61K 31/7004; A61K 36/899
USPC 514/25; 536/4.1

(57) ABSTRACT

The present invention relates to a use of a composition in the manufacture of a product for regulating a plurality of genes consisting of phosphoenolpyruvate carboxykinase-1, fructose-1,6-bisphosphatase, xenobiotic metabolism, low-density lipoprotein receptor, apolipoprotein-A1, superoxide dismutase-2, and catalase, characterised in that: the composition comprising acylated steryl glucoside.

### BACKGROUND OF THE INVENTION

1. Field of the Invention

This invention relates to a use of a composition in the manufacture of a product for regulating a plurality of genes more particularly to a composition comprising acylated steryl glucoside in the manufacture of a product for regulating a plurality of genes.

2. Description of Related Arts

Gluconeogenesis is a metabolic pathway that results in the production of glucose from non-carbohydrate carbon substrates such as lactate, glycerol, and glucogenic amino acids. High production of glucose may lead to a condition termed as hyperglycaemia and it may lead to condition such as diabetes mellitus to occur.

Oxidative stress represents an imbalance between the production of reactive oxygen species and a biological system's ability to readily detoxify the reactive intermediates or easily repair the resulting damage. All forms of life maintain a reducing environment within their cells. This reducing environment is preserved by enzymes that maintain the reduced state through a constant input of metabolic energy. Disturbances in this normal redox state can cause toxic effects through the production of peroxides and free radicals that damage all components of the cell, including proteins, lipids, and DNA. The reactive oxygen species include hydrogen peroxide (H₂O₂), hydroxyl radical (OH) and superoxide anion (O₂⁻). These oxidants can damage cells by starting chemical chain reactions such as lipid peroxidation, or by oxidising DNA or proteins. Damage to DNA can cause mutations and possibly cancer while damage to proteins causes enzyme inhibition, denaturation and protein degradation. In human, oxidative stress is involved in many diseases, such as diabetes, cancer, and cardiovascular diseases. Cells in human are protected against oxidative stress by an interacting network of antioxidant enzymes.

Xenobiotic metabolism refers to a various chemical reactions, called metabolic pathways that a living organism uses to alter chemicals that are not normally found in an organism as part of its natural biochemistry. These chemicals, known as xenobiotics, can include things such as poisons, drugs, and environmental pollutants. Xenobiotic metabolism is important for life as it allows an organism to neutralise and eliminate foreign toxins that would otherwise interfere with the chemical processes that keep it alive. The reactions involved in the metabolic pathways are of particular interest in medicine as part of drug metabolism and as a factor contributing to multidrug resistance in cancer chemotherapy. For example, diabetes mellitus comes along with a lot of health problems and so it is common to find diabetics on medications for hypertension, infections, diabetes mellitus, arrhythmias, anticoagulation etc. The diet and other drugs taken concurrently with medications could have a wide range of effects on the metabolism of drugs. A slow xenobiotic (drug) metabolism may result in higher efficacy of the drug in the patient's body. On the other hand, over expression of xenobiotic metabolism genes may cause faster metabolism of drugs therefore making the drugs less efficacious and possibly causing unwanted effects to the patient's body if the metabolite is the active form of the drug.

Low-density lipoprotein (LDL) is one of the major groups of lipoproteins that enable lipids like cholesterol and triglycerides to be transported within the water-based bloodstream. High LDL in the blood may lead to the progression of atherosclerosis and other cardiovascular diseases. High-density lipoprotein (HDL) enables lipids like cholesterol and triglycerides to be transported within the water-based bloodstream. HDL is often contrasted with LDL and HDL particles are able to remove cholesterol from within the arteries and transport it back to the liver for excretion or re-utilisation. Higher HDL-cholesterol seems to have fewer problems with cardiovascular diseases.

Therefore, there is a need to have an alternative solution to reduce gluconeogenesis, reduce generation of free radicals, reduce xenobiotic metabolism, and control cholesterol level by reducing LDL and increasing HDL in various diseases like diabetes mellitus type-2, cardiovascular disease, and other diseases related to oxidative stress. There is also a need to reduce the burden of these diseases since despite existing therapies, the conditions of the diseases still persist and the occurrence of the diseases seem to be increasing.

In nutrition, diet is the sum of food consumed by an organism. Dietary habits are the habitual decisions an individual makes when choosing what food to eat. Proper nutrition requires the proper ingestion and, equally important, the absorption of vitamins, minerals, and food energy in the form of carbohydrates, proteins, and fats. Dietary habits and choices play a significant role in health and mortality. Hence, it is important for a person to choose the right diet in order to maintain his health or to at least reduce the risk of getting diseases or infections. Therefore, there is a need to make use of daily diet that is able to function as a product to reduce gluconeogenesis, reduce generation of free radicals, reduce xenobiotic metabolism, and control cholesterol level by reducing LDL and increasing HDL, especially in an individual with certain diseases such as diabetes mellitus type-2, cardiovascular diseases, and other diseases related to oxidative stress.

A gene is a unit of hereditary in a living organism. It normally resides on some stretches of DNA and RNA that codes for a type of protein or for an RNA chain that has a function in the organism. Living things depend on genes, as they specify all proteins and functional RNA chains. Regulation of gene expression is termed to be the processes that cells and viruses use to regulate the way that the information in genes is turned into gene products. The interaction between genes and dietary components has been elucidated lately under the emerging field of Nutrigenomics. Different dietary components are now known to increase or decrease the risk of diseases through and increase or decrease in gene expression, and that interaction can be studied through different nutrigenomics tools. An increase or decrease in expression of a gene as studied by different nutrigenomics tools will therefore lead to an increase or decrease in production of the related protein the gene encodes for, ultimately leading to an effect. Therefore, specifying a treatment to the gene that expresses a particular enzyme (protein) could be useful in regulating genes involved in gluconeogenesis, oxidant system, xenobiotic metabolism, and LDL and HDL, in treating and preventing diabetes mellitus type-2 and other diseases related to oxidative stress.

US Patent Application No. 2008/0260873 A1 disclosed a use of lipid fraction in pre-germinated brown rice for prevention or improvement of diabetic neuropathy. Ingestion of the lipid fraction in the prior art provides an effect of improving diabetic neuropathy by increasing Na, K-ATPase activity or HTase activity decreased by diabetic neuropathy to a near normal level or an effect of preventing a decrease in motor nerve conduction velocity, within a whole-body, a tissue, a cell or body fluid of human or animals. However, the prior art did not mentioned the use of the lipid fraction in gene regulation. Therefore, there is a need for an alternative solution that is able to use a composition comprising the lipid fraction in regulating genes for treating and preventing diseases related to oxidative stress, particularly diabetes mellitus.

### SUMMARY OF INVENTION

It is an object of the present invention to provide a use of a composition comprising a compound in the manufacture of a product for regulating a plurality of genes.

It is also an object of the present invention to provide an alternative solution to regulate genes involved in gluconeogenesis in human or animal

It is yet another object of the present invention to provide an alternative solution to regulate genes involved in cholesterol metabolism in human or animal.

It is a further object of the present invention to provide an alternative solution to regulate genes involved in oxidative stress in human or animal.

It is also further object of the present invention to provide alternative solution to regulate genes involved in xenobiotic metabolism in human or animal.

It is yet another object of the present invention to provide the use of the composition in the manufacture of a pharmaceutical product or nutraceutical product.

Accordingly, these objectives may be achieved by following the teachings of the present invention. The present invention relates to a use of a composition in the manufacture of a product for regulating a plurality of genes consisting of phosphoenolpyruvate carboxykinase-1, fructose-1,6-bisphosphatase, xenobiotic metabolism, low-density lipoprotein receptor, apolipoprotein-A1, superoxide dismutase-2, and catalase, characterised in that: the composition comprising acylated steryl glucoside.

### BRIEF DESCRIPTION OF THE DRAWINGS

The features of the invention will be more readily understood and appreciated from the following detailed description when read in conjunction with the accompanying drawings of the preferred embodiment of the present invention, in which:

FIG. 1 is a pathway diagram in gluconeogenesis.

FIG. 2 shows a graph on fold increase in phosphoenolpyruvate carboxykinase-1(PEPCK-1) gene expression in diabetic rats.

FIG. 3 shows a graph on fold increase in fructose-1,6-bisphosphatase (F-1,6-BP) gene expression in diabetic rats.

FIG. 4 shows a graph on percentage change in expression of xenobiotic metabolism genes in rats.

FIG. 5 shows a graph on percentage change in low-density lipoprotein (LDL) in diabetic rats.

FIG. 6 shows a graph on fold increase in low-density lipoprotein receptor (LDLR) gene expression in diabetic rats.

FIG. 7 shows a graph on percentage change in high-density lipoprotein (HDL) in diabetic rats.

FIG. 8 shows a graph on fold increase in apolipoprotein-A1 (APO-A1) gene expression in diabetic rats.

FIG. 9 shows a pathway diagram in oxidative stress

FIG. 10 shows a graph on fold increase in superoxide dismutase-2 gene expression in rats.

FIG. 11 shows a graph on fold increase in catalase gene expression in rats.

### DETAILED DESCRIPTION OF THE INVENTION

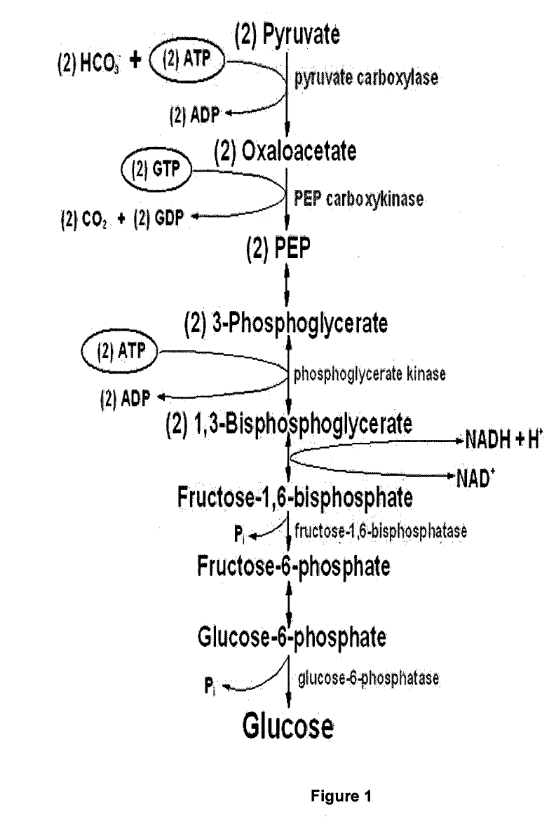

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting but merely as a basis for claims. It should be understood that the drawings and detailed description thereto are not intended to limit the invention to the particular form disclosed, but on the contrary, the invention is to cover all modification, equivalents and alternatives falling within the scope of the present invention as defined by the appended claims. As used throughout this application, the word “may” is used in a permissive sense (i.e., meaning having the potential to), rather than the mandatory sense (i.e., meaning must). Similarly, the words “include,” “including,” and “includes” mean including, but not limited to. Further, the words “a” or “an” mean “at least one” and the word “plurality” means one or more, unless otherwise mentioned. Where the abbreviations of technical terms are used, these indicate the commonly accepted meanings as known in the technical field. For ease of reference, common reference numerals will be used throughout the figures when referring to the same or similar features common to the figures. The present invention will now be described with reference to FIGS. 1-11.

The present invention relates to a use of a composition in the manufacture of a product for regulating a plurality of genes.

The use of the composition in the manufacture of the product for regulating the plurality of genes consisting of phosphoenolpyruvate carboxykinase-1, fructose-1,6-bisphosphatase, xenobiotic metabolism, apolipoprotein-A1, superoxide dismutase-2, and catalase, characterised in that the composition comprises acylated steryl glucoside.

In a preferred embodiment, the acylated steryl glucoside (ASG) is extracted from germinated brown rice. In a preferred embodiment, the acylated steryl glucoside is extracted from germinated brown rice by Folch method.

In a preferred embodiment, the composition comprising acylated steryl glucoside is in germinated brown rice.

In a preferred embodiment, the composition comprises acylated steryl glucoside and germinated brown rice.

In a preferred embodiment, the product comprises the composition and a pharmaceutically acceptable carrier, diluent, or an excipient. The product comprising the composition may be tablet, capsule, emulsion, powder, solution, suspension, emulsion, or the like.

In a preferred embodiment, the product comprises the composition and a nutraceutically acceptable carrier, preservatives, colouring, or flavourings. In a preferred embodiment, the product is food containing the composition, pellet containing the composition, drinks containing the composition or the like.

In a preferred embodiment, the composition is used in the manufacture of the product for down-regulating phosphoenolpyruvate carboxykinase-1 (PEPCK-1) gene. In a preferred embodiment, the composition is used in the manufacture of the product for down-regulating fructose-1,6-bisphosphatase gene. In a preferred embodiment, the composition is used in the manufacture of the product for reducing gluconeogenesis (refer to FIG. 1). More particularly, the composition may be used in the manufacture of the product for down-regulating PEPCK-1 gene or fructose-1,6-bisphosphate gene, for reducing gluconeogenesis.

PEPCK-1 gene is involved in gluconeogenesis, possibly in human or animal having diabetes mellitus type-2. Fructose-1,6-bisphosphatase gene is involved in gluconeogenesis, possibly in human or animal having diabetes mellitus type-2.

Referring to FIG. 1, over expression of phosphoenolpyruvate carboxykinase (PEPCK) enzyme may result in high production of glucose. Therefore, it may cause glucose level to be increased in the blood of the human or animal. The present invention promotes a solution to down-regulate the expression of PEPCK-1 gene so that the expression of PEPCK enzyme is controlled. When the expression of PEPCK enzyme is controlled, the production of phosphoenolpyruvate (PEP) may also be controlled or reduced, therefore resulting in the production of end product that is glucose to be reduced. Thus, glucose level in blood may be controlled or reduced to a normal range (fasting level of about 3.6 to 5.8 mmol/L in humans). The word ‘controlled’ herein describes that a parameter mentioned in this description is maintained at a range consistently without fluctuating to a level similar to controls not taking the product.

The present invention also control the expression of fructose-1,6-bisphosphatas enzyme by down-regulating the expression of fructose-1,6-bisphosphatase gene. As the expression of fructose-1,6-bisphosphatase enzyme is controlled or reduced by the down-regulation of fructose-1,6-bisphosphatase gene, the production of fructose-6-phosphate may also be controlled or reduced, and therefore resulting in reduced production of end product that is glucose (FIG. 1). Thus, glucose level in blood may be controlled or reduced to a range consistently without fluctuating to a level similar to controls not taking the product.

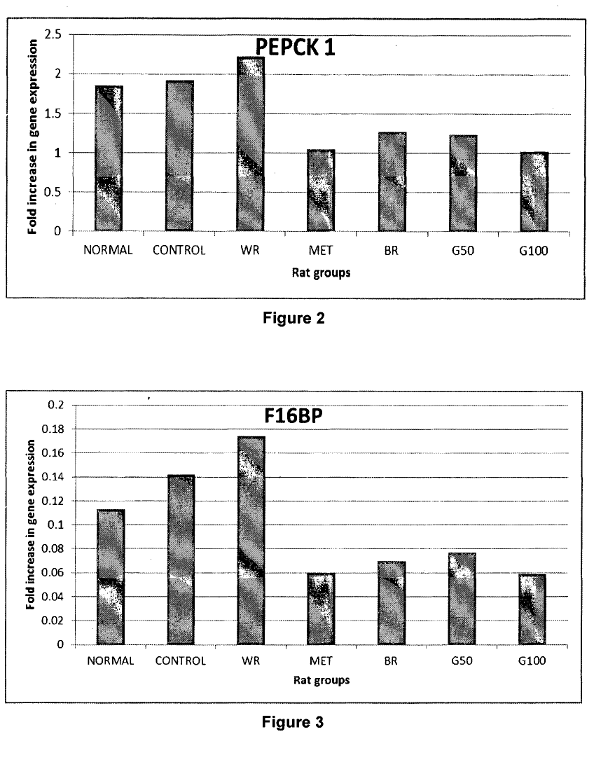

FIG. 2 shows a graph on fold increase in PEPCK-1 gene expression in diabetic (type-2) rats after being fed with white rice, metformin, brown rice, 50% germinated brown rice (G50), and 100% germinated brown rice (G100). FIG. 2 shows that G50 and G100 down-regulates the expression of PEPCK-1 gene with a lower fold increase in gene expression compared to normal rats, control rats, white rice-fed rats, metformin-treated rats, and brown rice-fed rats.

FIG. 3 shows a graph on fold increase fructose-1,6-bisphosphatase gene expression in diabetic (type-2) rats after being fed with white rice, metformin, brown rice, 50% germinated brown rice (G50), and 100% germinated brown rice (G100). FIG. 3 shows that in G50 and G100 there is down-regulation of the expression of fructose-1,6-bisphosphatase gene with a lower fold increase in gene expression compared to normal rats, control rats, white rice-fed rats, metformin-treated rats, and brown rice-fed rats.

In a preferred embodiment, the composition is used in the manufacture of the product for down-regulating xenobiotic metabolism genes. In a preferred embodiment, the composition is used in the manufacture of the product for reducing drug metabolism. The present invention promotes the down-regulation of xenobiotic metabolism genes which are responsible in the expression of xenobiotic metabolism enzymes. As the xenobiotic metabolism genes are down-regulated, the expression of xenobiotic metabolism enzymes are controlled or reduced, thus, normalising the metabolic pathways in the organism.

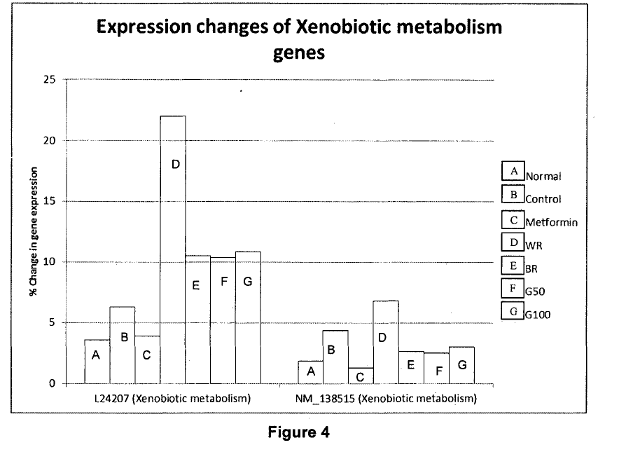

FIG. 4 shows a graph on percentage change in expression of xenobiotic metabolism genes in rats (grouped: normal rats, control rats, white rice-fed rats, metformin-treated rats, brown rice-fed rats, 50% germinated brown rice-fed rats, and 100% germinated brown rice-fed rats). NM_{—}138515 represents cytochrome P450, family 2, subfamily d, polypeptide 22 (Cyp2d22) and L24207 represents cytochrome p-450, family 3, subfamily A, polypeptide 1 (CYP3A1). FIG. 4 shows that in G50 and G100 there is down-regulation of the expression of xenobiotic metabolism with lower fold increase in gene expression compared to white rice-fed rats.

In a preferred embodiment, the composition comprises acylated steryl glucoside is used in the manufacture of the product for up-regulating low-density lipoprotein receptor gene. In a preferred embodiment, the composition is used in the manufacture of the product for decreasing blood levels of low density lipoprotein. The present invention may reduce the risk of atherosclerosis and other diseases related to high cholesterol level by promoting up-regulation of low-density lipoprotein receptor (LDLR) gene which expresses LDLR. LDLR may bind to LDL-cholesterol and internalised in a process known as endocytosis and prevent low-density lipoprotein (LDL) from diffusing around the membrane surface which occurs in all nucleated cells mainly in the liver of human or animal. This may therefore remove LDL from blood circulation. LDL-cholesterol herein is defined as the amount of cholesterol contained in LDL.

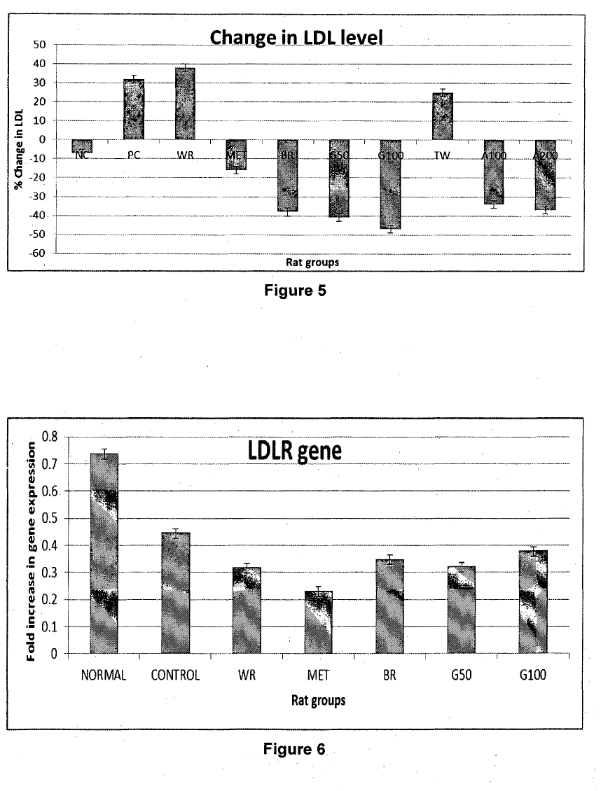

FIG. 5 shows a graph on percentage change in low-density lipoprotein (LDL) in diabetic rats. FIG. 5 shows that the LDL level in the diabetic rats is reduced in A100 (ASG in 100 mg/kg body weight), A200 (ASG in 200 mg/kg body weight), brown rice-fed rats, metformin-treated rats, G50-fed (50% germinated brown rice-fed) rats, and G100-fed (100% germinated brown rice-fed) rats compared to white rice-fed rats and tween-treated rats. FIG. 6 shows a graph on fold increase in low-density lipoprotein receptor (LDLR) gene expression in rats (grouped: normal rats, control rats, white rice-fed diabetic rats, metformin-treated diabetic rats, brown rice-fed diabetic rats, 50% germinated brown rice-fed diabetic rats, and 100% germinated brown rice-fed diabetic rats). FIG. 6 shows that in G50 and G100 there is up-regulation of the gene expression for LDLR gene with a higher fold increase compared to metformin-treated diabetic rats and white rice-fed diabetic rats.

In a preferred embodiment, the composition comprises acylated steryl glucoside is used in the manufacture of the product for up-regulating apolipoprotein-A1 gene. In a preferred embodiment, the composition is used in the manufacture of the product for increasing blood levels of high density lipoprotein. The present invention promotes the up-regulation of apolipoprotein-A1 (APO-A1) gene which enables expression of APO-A1 protein to remove cholesterol from the blood circulation.

FIG. 7 shows a graph on percentage change in high-density lipoprotein (HDL) in diabetic rats. FIG. 7 shows that the HDL level in diabetic rats is increased in brown-rice fed rats, G50-fed rats, G100-fed rats, and A200-fed (ASG in 200 mg/kg body weight) rats compared to white-rice fed rats and metformin-treated rats.

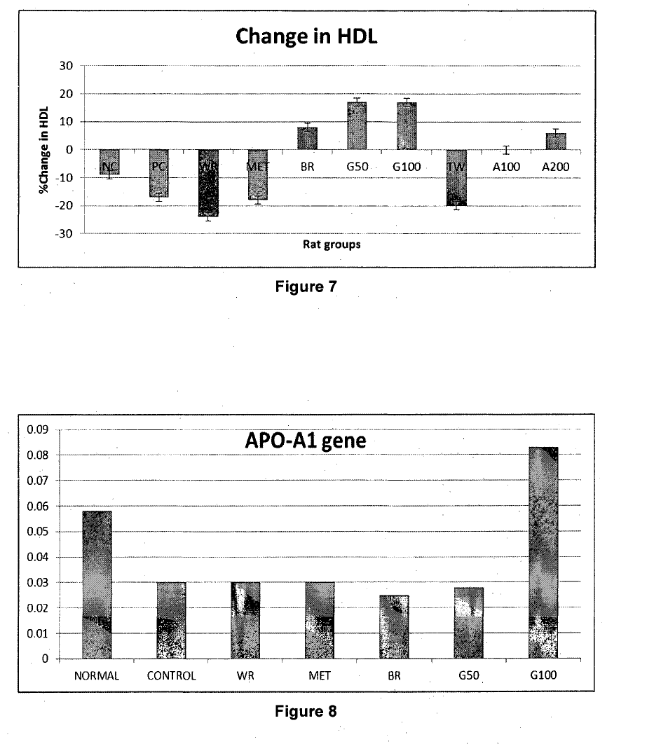

FIG. 8 shows graph on fold increase in apolipoprotein-A1 (APO-A1) gene expression in rats (grouped: normal rats, control rats, white rice-fed diabetic rats, metformin-treated diabetic rats, brown rice-fed diabetic rats, 50% germinated brown rice-fed diabetic rats, and 100% germinated brown rice-fed diabetic rats).

FIG. 8 shows that the up-regulation in APO-A1 gene has a higher fold increase in G100 compared to metformin-treated rats, white rice-fed rats, and brown rice-fed rats.

In a preferred embodiment, the composition is used in the manufacture of the product for up-regulating superoxide dismutase-2 gene. In a preferred embodiment, the composition is used in the manufacture of the product for up-regulating catalase gene. In a preferred embodiment, the composition is used in the manufacture of the product for reducing oxidative stress. More particularly, the composition may be used in the manufacture of the product for up-regulating superoxide dismutase-2 gene or catalase gene, for reducing oxidative stress.

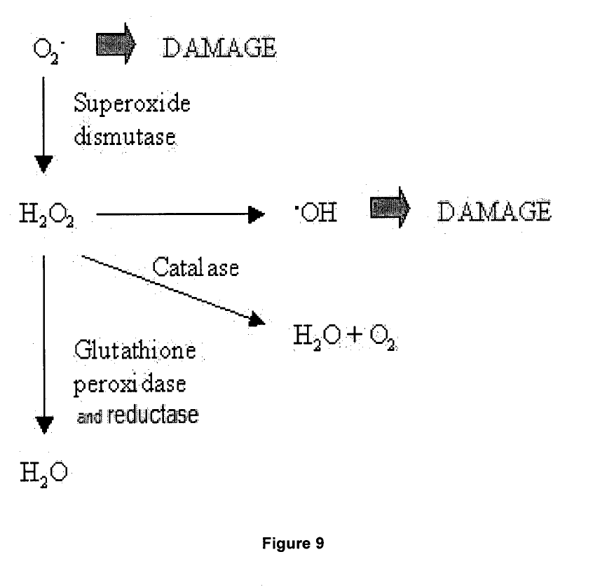

FIG. 9 shows that superoxide dismutase enzyme catalyse the breakdown of superoxide anion (a reactive oxygen species) into oxygen and hydrogen peroxide. The present invention promotes the up-regulation of superoxide dismutase-2 gene which increases the expression of superoxide dismutase enzyme, preferably superoxide dismutase-2 enzyme. The up-regulation of superoxide dismutase-2 gene may therefore increases the expression of superoxide dismutase enzyme, thus more superoxide anion are broken down to hydrogen peroxide.

The present invention also promotes the up-regulation of catalase gene which increases the expression of catalase enzyme. As the expression of catalase enzyme increases, more hydrogen peroxide (reactive oxygen species) may be reduced to oxygen and water.

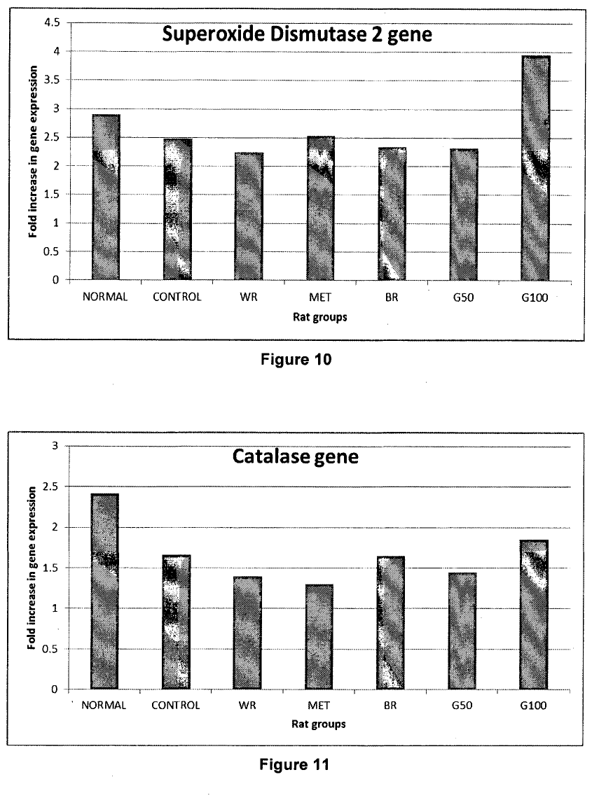

FIG. 10 shows a graph on fold increase in superoxide dismutase-2 gene expression in rats (grouped: normal rats, control rats, white rice-fed diabetic rats, metformin-treated diabetic rats, brown rice-fed diabetic rats, 50% germinated brown rice-fed diabetic rats, and 100% germinated brown rice-fed diabetic rats).

FIG. 10 shows that G100 have up-regulated superoxide dismutase-2 gene expression with a higher fold increase compared to brown rice-fed diabetic rats, metformin-treated diabetic rats, and white rice-fed diabetic rats.

FIG. 11 shows a graph on fold increase in catalase gene expression in rats (grouped: normal rats, control rats, white rice-fed diabetic rats, metformin-treated diabetic rats, brown rice-fed diabetic rats, 50% germinated brown rice-fed diabetic rats, and 100% germinated brown rice-fed diabetic rats). FIG. 11 shows that G100 have up-regulated superoxide dismutase-2 gene expression with a higher fold increase compared to brown rice-fed diabetic rats, metformin-treated diabetic rats, and white rice-fed diabetic rats.

A method for treatment or prophylaxis of a disease in a human or animal, characterised by administering 100-200 mg/kg body weight of acylated steryl glucoside to the human or animal. In a preferred embodiment, said disease is caused by or linked to oxidative stress.

In a preferred embodiment, the present invention may be applicable to human or animal having diseases related to oxidative stress as shown in table 1. In a preferred embodiment, the present invention can be used to a human or animal having diabetes mellitus type-2, which suffered from high blood glucose level, high cholesterol level, high oxidants level, and high xenobiotic metabolism.

TABLE 1
Diseases related to Oxidative Stress

| No | Disease | Example |
|---|---|---|
| 1 | Neurodegenerative | Parkinson disease |
| | | Alzheimer disease |
| | | Multiple Sclerosis |
| | | Schizophrenia |
| | | Dementia |
| | | Huntington's disease |
| 2 | Aging | Arthritis |
| | | Diabetes |
| | | Osteoarthritis cataract |
| | | Macular Degeneration |
| | | Prostate problems |
| 3 | Cancer | Prostate cancer |
| | | Breast cancer |
| | | Lung cancer |
| | | Colorectal cancer |
| | | Bladder cancer |
| | | Uterine cancer |
| | | Ovarian cancer |
| | | Lymphoma |
| | | Skin cancer |
| | | Stomach cancer |
| | | Liver cancer & others |
| | | Wasting disease |
| 4 | Liver | Toxic hepatitis |
| | | Viral hepatitis (A, B, C) |
| | | Chronic hepatitis |
| | | Cirrhosis |
| 5 | Lung | Asthma emphysema |
| | | Pneumonia |
| | | Bronchitis (Chronis and acute) |
| | | Cystic fibroses |
| | | Pulmonary fibroses |
| | | Chronic obstructive pulmonary disease (COPD) |
| | | Adult respiratory distress syndrome (ARDS) |
| 6 | Cardiovascular | Arteriosclerosis and its consequences |
| | | Heart failure |
| | | Heart attack |
| | | Kidney failure |
| | | High blood pressure |
| | | Stroke |
| | | Impaired circulation |
| | | Heart disease |
| | | Cholesterol and plaque formation |
| | | Reperfusion injury |
| 7 | Digestive | Inflammatory bowel disease |
| | | Ulcerative colitis |
| | | Crohn's disease |
| | | Gastritis |
| | | Stomach cancer |
| | | Pancreatitis |
| | | Peptic ulcer |
| 8 | Kidney failure and | Kidney failure |
| | dialysis | Renal toxicity |
| | | Oxidative stress from dialysis |
| 9 | Infectious diseases | Viral infection HIV and AIDS |
| | and Immunology | Toxic hepatitis and cirrhosis |
| | | Viral hepatitis (type A, B, &C) herpes |
| | | Common cold |
| | | Bacterial infection |
| | | Chronic fatigue syndrome |
| | | Certain autoimmune dysfunction |
| 10 | Skin disorders | Psoriases |
| | | Eczema |
| | | SLE (Lupus) |
| | | Vasculitis |
| | | Polymyositis |
| | | Mycosis fungoides |
| | | Scleroderma |
| | | Pemhigoid |
| | | Atopic dermatitis |
| | | Contact dermatitis |
| | | Sebborrheic dermatitis |
| | | Dermatitis herpetiformis |
| | | Acne conglobata |
| | | Acne vularis |
| | | UV radiation skin damage |
| 11 | Eye, ear, nose, | Cataract |
| | throat and teeth | Glaucoma |
| | | Macular degeneration |
| | | Hearing loss |
| | | Ear infection |
| | | Sinusitis |
| | | Periodontal disease |
| | | Nose, mouth and throat disease |
| 12 | Pregnancy, lactation | Pre-eclampsia |
| | and childbirth | Eclampsia |
| | | Hypertension |
| | | Diabetes |
| 13 | Male problems | Prostate enlargement |
| | | Prostate cancer |
| | | Balding and hair loss |
| | | Male infertility |
| 14 | Exercise and athletic | Over-training syndrome and the related |
| | performance | oxidative stress |

Method of Germinating Brown Rice

In a preferred embodiment, the ASG in the present invention is extracted from germinated brown rice of is in germinated brown rice, wherein the germinated brown rice is germinated by a preferred method. In the preferred method, the brown rice can be germinated by firstly washing the brown rice in water at a temperature between 20-40° C. for 4-8 hours to produce washed brown rice; then immersing the washed brown rice in 0.1% (v/v) sodium hypochlorite for 30 minutes to produce immersed brown rice; followed by spraying immersed brown rice with water intermittently at 4-6 hours interval until sprouts appeared to produce sprouted brown rice; and then dried at 60° C. for 20 minutes to produce germinated brown rice. In a preferred embodiment, the germinated brown rice in the present invention has the composition comprising of gamma aminobutyric acid content ranging from 50-200 mg/g; gamma oryzanol content ranging from 0.007-0.01 mg/g; phytic acid content ranging from 0.3-0.6 mg/g; ferulic acid content ranging from 50-2000 mg/g; total dietary fibre content ranging from 6-11%; antioxidant activity ranging from 73-84%, and acylated steryl glucoside content ranging from 0.2-1.0 mg/g. In a preferred embodiment, the germinated brown contains acylated steryl glucoside and the composition in the germinated brown rice is used in the manufacture of the product.

Although the present invention has been described with reference to specific embodiments, also shown in the appended figures, it will be apparent for those skilled in the art that many variations and modifications can be done within the scope of the invention as described in the specification and defined in the following claims.

I/We claim:

1. A use of a composition in the manufacture of a product for regulating a plurality of genes consisting of phosphoenolpyruvate carboxykinase-1, fructose-1,6-bisphosphatase, xenobiotic metabolism genes, low-density lipoprotein receptor, apolipoprotein-A1, superoxide dismutase-2, and catalase, characterised in that the composition comprises acylated steryl glucoside.

2. A use of a composition in the manufacture of a product for regulating a plurality of genes, according to claim 1, wherein the acylated steryl glucoside is extracted from germinated brown rice.

3. A use of a composition in the manufacture of a product for regulating a plurality of genes, according to claim 1, wherein the composition comprising acylated steryl glucoside is in germinated brown rice.

4. A use of a composition in the manufacture of a product for regulating a plurality of genes, according to claim 1, wherein the composition comprises acylated steryl glucoside and germinated brown rice.

5. A use of a composition in the manufacture of a product for regulating a plurality of genes, according to claim 1, wherein the product comprises the composition and a pharmaceutically acceptable carrier, diluent, or excipient.

6. A use of a composition in the manufacture of a product for regulating a plurality of genes, according to claim 1, wherein the product comprises the composition and a nutraceutically acceptable carrier, preservatives, colouring, or flavourings.

7. A use of a composition in the manufacture of a product for regulating a plurality of genes, according to claim 1, wherein the composition is used in the manufacture of the product for down-regulating phosphoenolpyruvate carboxykinase-1 gene.

8. A use of a composition in the manufacture of a product for regulating a plurality of genes, according to claim 1, wherein the composition is used in the manufacture of the product for down-regulating fructose-1,6-bisphosphatase gene.

9. A use of a composition in the manufacture of a product for regulating a plurality of genes, according to claim 1, wherein the composition is used in the manufacture of the product for reducing gluconeogenesis.

10. A use of a compound in the manufacture of a product for regulating a plurality of genes, according to claim 1, wherein the composition is used in the manufacture of the product for down-regulating xenobiotic metabolism genes.

11. A use of a composition in the manufacture of a product for regulating a plurality of genes, according to claim 1, wherein the composition is used in the manufacture of the product for reducing drug metabolism.

12. A use of a composition in the manufacture of a product for regulating a plurality of genes, according to claim 1, wherein the composition comprises acylated steryl glucoside is used in the manufacture of the product for up-regulating low-density lipoprotein receptor gene.

13. A use of a composition in the manufacture of a product for regulating a plurality of genes, according to claim 1, wherein the composition is used in the manufacture of the product for decreasing blood levels of low density lipoprotein.

14. A use of a composition in the manufacture of a product for regulating a plurality of genes, according to claim 1, wherein the composition comprises acylated steryl glucoside is used in the manufacture of the product for up-regulating apolipoprotein-A1 gene.

15. A use of a composition in the manufacture of a product for regulating a plurality of genes, according to claim 1, wherein the composition is used in the manufacture of the product for increasing blood levels of high density lipoprotein.

16. A use of a composition in the manufacture of a product for regulating a plurality of genes, according to claim 1, wherein the composition is used in the manufacture of the product for up-regulating superoxide dismutase-2 gene.

17. A use of a composition in the manufacture of a product for regulating a plurality of genes, according to claim 1, wherein the composition is used in the manufacture of the product for up-regulating catalase gene.

18. A use of a composition in the manufacture of a product for regulating a plurality of genes, according to claim 1, wherein the composition is used in the manufacture of the product for reducing oxidative stress.
